(19) United States
(12) Patent Application Publication
(10) Pub. No.: US 2026/0364791 A1
(43) Pub. Date: Nov. 19, 2026

(54) SYSTEMS AND METHOD FOR DISSIPATING KINETIC ENERGY IN CONTROLLILNG A REPOSITIONABLE STRUCTURE

(71) Applicant: INTUITIVE SURGICAL OPERATIONS, INC., Sunnyvale, CA (US)

(72) Inventors: Amin ZEIAEE, Los Gatos, CA (US); Goran A. LYNCH, Oakland, CA (US)

(21) Appl. No.: 19/135,346

(22) PCT Filed: Dec. 5, 2023
(86) PCT No.: PCT/US2023/082598

§ 371 Date: Jun. 3, 2025

Related U.S. Application Data
(60) Provisional application No. 63/430,416, filed on Dec. 6, 2022.

(51) Int. Cl. B25J 9/16

(52) U.S. Cl. CPC B25J 9/1633; B25J 9/1689

(57) ABSTRACT

Systems and methods for dissipating kinetic energy in a repositionable structure include a repositionable structure having a joint, an actuator configured to control motion of the joint, and an energy dissipative device, where engaging the energy dissipative device dissipates a kinetic energy of the joint. A control system is coupled to the repositionable structure. The control system is configured to determine a target motion for the joint; determine, based on at least the target motion, whether to concurrently actuate the actuator and engage the energy dissipative device such that the energy dissipative device dissipates the kinetic energy of the joint while the actuator drives motion of the joint; and in response to the determination to concurrently actuate the actuator and engage the energy dissipative device.

### RELATED APPLICATIONS

This application claims the benefit of U.S. Provisional Application No. 63/430,416, filed Dec. 6, 2022, and entitled “Systems and Methods for Dissipating Kinetic Energy in Controlling a Repositionable Structure,” which is incorporated by reference herein.

### TECHNICAL FIELD

The present disclosure relates generally to computer-assisted systems and more particularly to dissipating kinetic energy in controlling a repositionable structure of a computer-assisted system.

### BACKGROUND

Computer-assisted systems are often used to perform or assist procedures in a workspace. In an example computer-assisted system with teleoperation, an operator at a user input system manipulates a leader device (e.g., an input device configured to accept commands for a follower device) to cause motions of a follower device (e.g., a manipulating assembly that can be teleoperated, and comprising a repositionable structure with or without a supported instrument). In an example, motions of the leader device relative to an operator frame of reference are used to determine corresponding motion commands for the follower device relative to a field of view of an imaging device.

By design, a repositionable structure often has high structural rigidity to achieve a high degree of kinematic accuracy. With such repositionable structures, it is common to enforce force or torque limits to the actuation of actuators of the joints of the repositionable structure. Force or torque limits can help to reduce the required size of joint actuators, reduce wear and tear on drive trains, and reduce forces or torques that can be exerted by the repositionable structure to other devices, structures in a workspace (e.g., parts of patient anatomy), personnel, and/or the like. However, the enforcement of force and torque limits also reduces the ability of the joint actuators to rapidly change a configuration of the repositionable structure.

Accordingly, improved techniques for selecting and managing force and torque limits and achieving desired control performance in the presence of force and torque limits in the joints of a repositionable structure are desired.

### SUMMARY

Consistent with some embodiments, a computer-assisted system includes a repositionable structure. The repositionable structure includes a first joint, a first actuator configured to control motion of the first joint, and a first energy dissipative device, where engaging the first energy dissipative device dissipates a kinetic energy of the first joint. The computer-assisted system further includes a control system coupled to the repositionable structure. The control system is configured to determine a first target motion for the first joint; determine, based on at least the first target motion, whether to concurrently actuate the first actuator and engage the first energy dissipative device such that the first energy dissipative device dissipates the kinetic energy of the first joint while the first actuator drives motion of the first joint; and in response to the determination to concurrently actuate the first actuator and engage the first energy dissipative device, concurrently actuate the first actuator and engage the first energy dissipative device.

Consistent with some embodiments, a method includes determining, by a control system, a first target motion for a first joint of a repositionable structure; determining, by the control system based on at least the first target motion, whether to concurrently actuate a first actuator and engage a first energy dissipative device such that the first energy dissipative device dissipates kinetic energy of the first joint while the first actuator drives motion of the first joint; and in response to the determining to concurrently actuate the first actuator and engage the first energy dissipative device, concurrently actuating, by the control system, the first actuator and engaging, by the control system, the first energy dissipative device.

Consistent with some embodiments, one or more non-transitory machine-readable media include a plurality of machine-readable instructions which when executed by a processor system are adapted to cause the processor system to perform any of the methods described herein.

### BRIEF DESCRIPTION OF THE DRAWINGS

FIG. 1 is a diagram of a computer-assisted system in accordance with one or more embodiments.

FIG. 2 is a diagram of a computer-assisted system in accordance with one or more embodiments.

FIG. 3 illustrates a control system for controlling motion of a joint and dissipating kinetic energy associated with the motion of the joint in accordance with one or more embodiments.

FIG. 4 is a flow diagram of method steps for dissipating kinetic energy associated with motion of a joint in accordance with one or more embodiments.

FIG. 5 is a flow diagram of method steps for applying haptic feedback to a joint in accordance with one or more embodiments.

In the figures, elements having the same designations have the same or similar functions.

### DETAILED DESCRIPTION

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

As discussed above, it is common to enforce force or torque limits for the joint actuators used to control a repositionable structure. These force and torque limits allow smaller joint actuators to be used, reduce wear and tear on drive trains, and reduce forces or torques that can be exerted by the repositionable structure to other objects. These force and torque limits also reduce how rapidly a configuration of the repositionable structure can be changed. For example, the joints of the repositionable structure that are used to control a larger inertial load or a larger mass, such as joints that are located in more proximal portions of the repositionable structure (e.g., that are located more proximally in a kinematic series) are often required to exert more force or torque to control the repositionable structure. Thus, selection of the force or torque limits for the joint actuators can present some challenges. For example, while lower force or torque limits can be useful for reducing the required size of joint actuators, reducing wear and tear on drive trains, and reducing forces that can be exerted by the repositionable structure, these force and torque limits can be counterproductive for achieving desired control performance (e.g. when a motion of the joint is to be arrested quickly).

As described in further detail below, a solution to this problem is to use an energy dissipative device, such as a brake or a variable damper, to assist a joint actuator to slow down motion of a corresponding joint. More specifically, to decelerate a joint, the joint actuator is used, the energy dissipative device is used, or both are used, to reduce the speed of the joint. Reducing the speed of the joint reduces the associated kinetic energy. This advantageously allows a smaller joint actuator to be used and/or reduces wear and tear on the joint actuator and the drive train between the joint actuator and the joint, and the like, while still allowing the joint to be actuated at a higher speed and then quickly slowed down, compared to other systems with similar actuators and drive trains.

This disclosure describes various elements (such as systems and devices, and portions of systems and devices) with examples in three-dimensional space. In such examples, the term “position” refers to the location of an element or a portion of an element in a three-dimensional space (e.g., three degrees of translational freedom along Cartesian x-, y-, and z-coordinates). Also in such examples, the term “orientation” refers to the rotational placement of an element or a portion of an element (three degrees of rotational freedom—e.g., roll, pitch, and yaw). Other examples may encompass other dimensional spaces, such as two-dimensional spaces. As used herein, the term “pose” refers to the position, the orientation, or the position and the orientation combined, of an element or a portion of an element. As used herein, and for an element or portion of an element (e.g., part or all of a computer-assisted system, a repositionable arm, a series of links and joints, personnel, etc.), the term “proximal” for elements in a kinematic series refers to a direction toward the base of the kinematic series, and the term “distal” refers to a direction away from the base along the kinematic series.

As used herein, the term “pose” refers to the multi-degree of freedom (DOF) spatial position and orientation of a physical element. The pose can be expressed in a coordinate system of interest, which can be referenced to the world, attached to the physical element, or referenced to some other appropriate reference. In general, a pose includes a pose variable for each of the DOFs in the pose. For example, a full 6-DOF pose for a frame of reference or a rigid body would include 6 pose variables corresponding to the 3 positional DOFs (e.g., x, y, and z) and the 3 orientational DOFs (e.g., roll, pitch, and yaw). (A non-rigid body may have additional DOFs, such as internal DOFs within the body, or different DOFs associated with different portions of the body that can translate or rotate or be deformed relative to each other.) In this example, A 3-DOF position-only pose would include only pose variables for the 3 positional DOFs. Similarly, a 3-DOF orientation-only pose would include only pose variables for the 3 rotational DOFs. Further, a velocity of the pose captures the change in pose over time (e.g., a first derivative of the pose). For the full 6-DOF pose example above, the velocity would include 3 translational velocities and 3 rotational velocities. Poses with other numbers of DOFs would have a corresponding number of velocities translational and/or rotational velocities.

Aspects of this disclosure are described in reference to electronic systems and computer-assisted systems, which may include systems and devices that are teleoperated, remote-controlled, autonomous, semiautonomous, manually manipulated, and/or the like. Example computer-assisted systems include those that comprise robots or robotic devices. Further, aspects of this disclosure are described in terms of an embodiment using a medical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

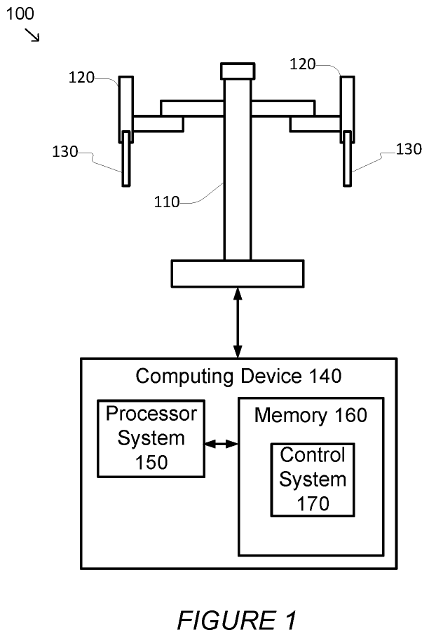

FIG. 1 is a diagram of a computer-assisted system 100 in accordance with one or more embodiments. As shown in FIG. 1, the computer-assisted system 100 includes, without limitation, a manipulating assembly 110 with one or more repositionable structures 120. In the example of FIG. 1, the repositionable structure(s) are shown as manipulator arms comprising a plurality of links coupled by one or more joints. Each of the one or more repositionable structures 120 can support one or more instruments 130. In some examples, the manipulating assembly 110 comprises a computer-assisted surgical assembly. Examples of medical instruments include surgical instruments for interacting with tissues, imaging devices, sensing devices, and/or the like. In some examples, the instruments 130 includes end effectors that are capable of, but are not limited to, performing, gripping, retracting, cauterizing, ablating, suturing, cutting, stapling, fusing, sealing, etc., and/or combinations thereof.

In a teleoperation example, the manipulating assembly 110 is further communicatively coupled by wired or wireless connection to a user input system (not shown). The user input system includes one or more input controls, also referred to herein as input devices, for operating the manipulating assembly 110, the one or more repositionable structures 120, and/or the instruments 130. In some examples, the one or more input controls also include corresponding repositionable structures that are separate and different from the one or more repositionable structures 120. The corresponding repositionable structures of the one or more input controls can include kinematic series of links and one or more joint(s), one or more actuators for driving portions of the input control(s), robotic manipulators, levers, pedals, switches, keys, knobs, triggers, and/or the like.

In examples supporting external manipulation, the input controls can be located at the repositionable structure. As a specific example, the input controls can comprise joint sensors that detect joint deflection, and the computer-assisted system is configured to process certain joint deflections to be commands to move the joint.

The manipulating assembly 110 of FIG. 1 is coupled to a computing device 140 via an interface. The interface can be wired and/or wireless, and can include one or more cables, fibers, connectors, and/or buses and can further include one or more networks with one or more network switching and/or routing devices. Operation of the computing device 140 is controlled by a processor system 150. Processor system 150 can include one or more central processing units, multi-core processors, microprocessors, microcontrollers, digital signal processors, field programmable gate arrays (FPGAs), application specific integrated circuits (ASICs), graphics processing units (GPUs), tensor processing units (TPUs), and/or the like in the computing device 140. The computing device 140 can be implemented as a stand-alone subsystem and/or board added to a computing device or as a virtual machine. In some embodiments, the computing device 140 is included as part of the user input system and/or the manipulating assembly 110, and/or is operated separately from, and in coordination with, the user input system and/or the manipulating assembly 110.

As one example, the manipulating assembly 110, the user input system, and/or the computing device 140 can correspond to the patient side cart, the surgeon console, and the processing units and associated software of da Vinci® Surgical System commercialized by Intuitive Surgical, Inc. of Sunnyvale, California. In some embodiments, manipulating assemblies with other configurations, such as fewer or more repositionable structures, different user input systems or input controls, different repositionable structure hardware, and/or the like, comprise the computer-assisted system 100.

The memory 160 is used to store software executed by the computing device 140 and/or one or more data structures used during operation of the computing device 140. The memory 160 can include one or more types of machine-readable media. Some common forms of machine-readable media can include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip, or cartridge, and/or any other medium from which a processor or computer is adapted to read.

As shown in the example of FIG. 1, the memory 160 includes a control system 170 that is used to support autonomous, semiautonomous, and/or teleoperated control of the manipulating assembly 110. The control system 170 can include one or more application programming interfaces (APIs) for receiving position, motion, force, torque, and/or other sensor information from the manipulating assembly 110, the repositionable structures 120, and/or the instruments 130, for sharing position, motion, force, torque, and/or collision avoidance information with other control units regarding other devices, and/or planning and/or assisting in the planning of motion for the manipulating assembly 110 (such as motion of the repositionable structures 120), and/or the instruments 130. In some examples, the control system 170 further supports autonomous, semiautonomous, and/or teleoperated control of the manipulating assembly 110 and/or the instruments 130 during the performance of various tasks. And although the control system 170 is depicted as a software application, the control system 170 can optionally be implemented using hardware, software, and/or a combination of hardware and software.

In a teleoperation example for computer-assisted system 100, an input control comprises a leader device (also called a “master” device in industry), and the manipulating assembly 110 or a repositionable structure 120 (either supporting or not supporting an instrument 130) comprises a follower device (also called a “slave” device in industry). An operator can use the one or more input controls to command motion of the manipulating assembly 110, such as by commanding motion of one or more repositionable structures 120 and/or instruments 130 by moving the one or more input controls, in a leader-follower configuration. The leader-follower configuration is a type of teleoperation configuration and is sometimes called a master-slave configuration in industry. Although not shown in FIG. 1, the one or more input controls can also include corresponding repositionable structures that are separate and different from the one or more repositionable structures 120.

In some medical embodiments, the computer-assisted system 100 can be found in a clinic, diagnostic facility, an operating room, an interventional suite, or other medical environment. Although the computer-assisted system 100 is shown comprising one manipulating assembly 110 with two repositionable structures 120, each supporting a corresponding instrument 130, one of ordinary skill would understand that the computer-assisted system 100 can include any number of manipulating assemblies, each manipulating assembly can comprise one or more repositionable structures, and each repositionable structure can support one or more instruments, and that all of these elements may be similar or different in design from that specifically depicted in these figures. In some examples, each of the manipulating assemblies can include fewer or more repositionable structures, and/or support fewer or more instruments, than specifically depicted in these figures.

In some implementations, each of the one or more repositionable structures 120 comprises a plurality of joints, such as drivable joints. Each of the drivable joints can be driven by one or more actuators to move the drivable joint, and thus move components physically coupled to the drivable joint. The one or more actuators can be used to accelerate or otherwise speed up, decelerate or otherwise slow down, or hold the respective drivable joint in place. In addition, each of the drivable joints can further be controlled using one or more energy dissipative devices, such as a brake or a damper. The one or more energy dissipative devices can be used to reduce kinetic energy in the drivable joint by slowing down the speed of the drivable joint. The one or more actuators, the one or more energy dissipative devices, or both the actuator(s) and the energy dissipative device(s) can be used to slow down (and reduce the speed of) the drivable joint.

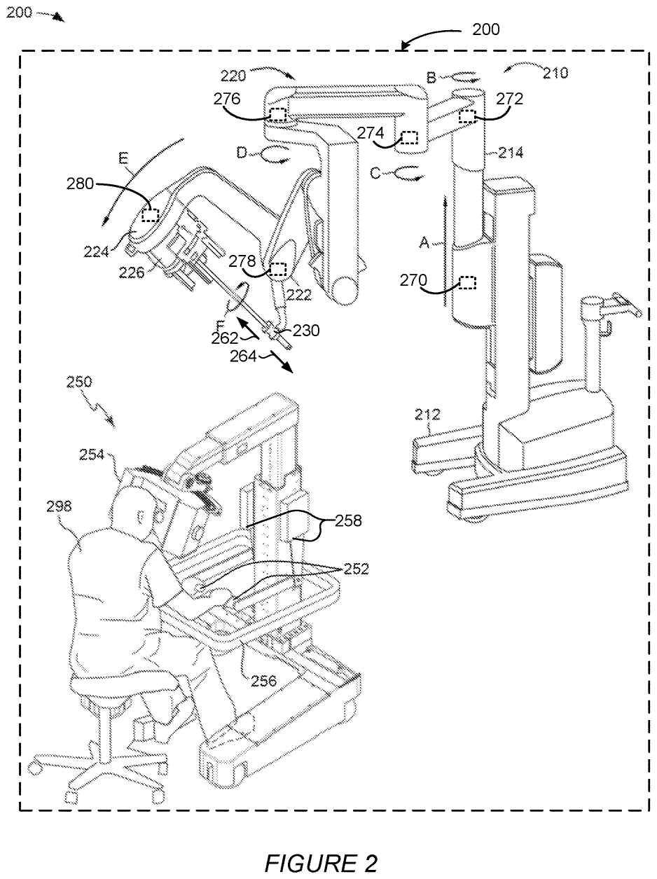

FIG. 2 is a diagram of a computer-assisted system 200 in accordance with one or more embodiments. The computer-assisted system 200, in the example of FIG. 2, includes, without limitation, a repositionable structure shown as a manipulating assembly 210, and a user input system 250. In a teleoperation scenario, an operator 298 uses the user input system 250 to operate the manipulating assembly 210, such as in a leader-follower configuration. In the leader-follower configuration for the example of FIG. 1, a component of the user input system 250 (e.g., an input control) is the leader, and a portion of the manipulating assembly 210 (e.g., a manipulator arm or other repositionable structure) is the follower.

The manipulating assembly 210 can be used to introduce a set of instruments into a work site through a single port 230 (e.g., using a cannula as shown) inserted in an aperture. In a medical scenario, the work site can be on or within a body of a patient, and the aperture can be a minimally invasive incision or a natural body orifice. The port 230 can be free-floating, held in place by a fixture separate from the manipulating assembly 210, or held by a linkage 222 or other part of the manipulating assembly 210. The linkage 222 can be coupled to additional joints and links 214, 220 of the manipulating assembly 210, and these additional joints and links 214, 220 can be mounted on a base 212. The linkage 222 can further include a manipulator-supporting link 224 located in a proximal direction 262 to the port 230. A set of manipulators 226 located in the proximal direction 262 to the port 230 can couple to the manipulator-supporting link 224. The repositionable structure that can be moved to follow commands from the user input system 250 can include one or more of any of the following: the linkage 222, additional joints and links 214, 220, base 212, manipulator-supporting link 224, and/or any additional links or joints coupled to the foregoing joints or links. Each of the manipulators 226 can include a carriage (or other instrument-coupling link) configured to couple to an instrument, and each of the manipulators 226 can include one or more joint(s) and/or link(s) that can be driven to move the carriage. For example, a manipulator 226 can include a prismatic joint that, when driven, linearly moves the carriage and any instrument(s) coupled to the carriage. This linear motion can be along (parallel to) an insertion axis that extends in a distal direction 264 to and through port 230.

The additional joints and additional links 214, 220 can be used to position the port 230 at the aperture or another position. FIG. 2 shows a prismatic joint for vertical adjustment (as indicated by arrow “A”) and a set of rotary joints for horizontal adjustment (as indicated by arrows “B” and “C”) that can be used to translate a position of the port 230. The linkage 222 is used to pivot the port 230 (and the instruments disposed within the port at the time) in yaw, pitch, and roll angular rotations about a remote center of motion (RCM) located in proximity to port 230 as indicated by arrows D, E, and F, respectively, without translating the RCM.

Actuation of the degrees of freedom provided by joint(s) of the instrument(s) (not shown) can be provided by actuators disposed in, or whose motive force (e.g., linear force or rotary torque) is transmitted to, the instrument(s). Examples of actuators include rotary motors, linear motors, solenoids, and/or the like. The actuators can drive transmission elements in the manipulating assembly 210 and/or in the instruments to control the degrees of freedom of the instrument(s). For example, the actuators can drive rotary discs of the manipulator that couple with drive elements (e.g., rotary discs, linear slides) of the instrument(s), where driving the driving elements of the instruments drives transmission elements in the instrument that couple to move the joint(s) of the instrument, or to actuate some other function of the instrument, such as a degree of freedom of an end effector. Accordingly, the degrees of freedom of the instrument(s) can be controlled by actuators that drive the instrument(s) in accordance with control signals. The control signals can be determined to cause instrument motion or other actuation as determined automatically by the system, as indicated to be commanded by movement or other manipulation of the input controls, or any other control signal. Furthermore, appropriately positioned sensors, e.g., encoders, potentiometers, and/or the like, can be provided to enable measurement of indications of the joint positions, or other data that can be used to derive joint position, such as joint velocity. The actuators and sensors can be disposed in, or transmit to or receive signals from, the manipulator(s) 226. Techniques for manipulating multiple instruments in a computer-assisted system are described more fully in Patent Cooperation Treaty Patent Application No. PCT/US2021/047374, filed Aug. 24, 2021, and entitled “METHOD AND SYSTEM FOR COORDINATED MULTIPLE-TOOL MOVEMENT USING A DRIVABLE ASSEMBLY,” which is incorporated herein by reference.

While a particular configuration of the manipulating assembly 210 is shown in FIG. 2, those skilled in the art will appreciate that embodiments of this disclosure can be used with any design of manipulating assembly or other repositionable structure. In some examples, a manipulating assembly can have any number and any types of degrees of freedom, can be configured to couple or not couple to an entry port, can optionally use a port other than a cannula, such as a guide tube, and/or the like. In some examples, the manipulating assembly 210 can also include an arrangement of links and joints that does not provide a remote center of motion.

In the example shown in FIG. 2, the user input system 250 includes one or more input controls 252 configured to be operated by the operator 298. In the example shown in FIG. 2, the one or more input controls 252 are supported by corresponding repositionable structures 258 allowing an operator to move the one or more input controls in various directions and/or degrees of freedom. The one or more input controls 252 are contacted and manipulated by the hands of the operator 298, with one input control 252 for each hand. Examples of such hand-input-devices include any type of device manually operable by human user, e.g., joysticks, trackballs, button clusters, and/or other types of haptic devices typically equipped with multiple degrees of freedom. Position, force, and/or tactile feedback devices (not shown) can be employed to transmit position, force, and/or tactile sensations from the instruments back to the hands of the operator 298 through the input controls 252.

The input controls 252 are supported by the user input system 250 using respective repositionable structures 258 that can include any number of joints and links. As shown, the input controls 252 are mechanically grounded via the respective repositionable structures 258, however, in other implementations can be mechanically ungrounded. An ergonomic support 256 can be provided in some implementations; for example, FIG. 2 shows an ergonomic support 256 including forearm rests on which the operator 298 can rest his or her forearms while manipulating the input controls 252. In some examples, the operator 298 can perform tasks at a work site near the manipulating assembly 210 during a procedure by controlling the manipulating assembly 210 using the input controls 252.

A display unit 254 is included in the user input system 250. The display unit 254 can display images for viewing by the operator 298. The display unit 254 can provide the operator 298 with a view of the worksite with which the manipulating assembly 210 interacts. The view can include stereoscopic images or three-dimensional images to provide a depth perception of the worksite and the instrument(s) of the manipulating assembly 210 in the worksite. The display unit 254 can be moved in various degrees of freedom to accommodate the viewing position of the operator 298 and/or to provide control functions. Where a display unit (such as the display unit 254 is also used to provide control functions, such as to command the manipulating assembly 210, the display unit also includes an input control (e.g., another input control 252).

Similar to computer-assisted system 100, computer-assisted system 200 and manipulating assembly 210 can optionally include a first joint set of drivable joints that are by mechanical configuration or software design constrained to produce motion that does not translate a remote center of motion (an RCM) and a second set of joints that can move the RCM.

In some examples, the repositionable structure includes a base manipulator and multiple instrument manipulators coupled to the base manipulator. In some examples, the repositionable structure includes a single instrument manipulator and no serial coupling of manipulators. In some examples, the repositionable structure includes a single instrument manipulator coupled to a single base manipulator. In some examples, the computer-assisted system can include a moveable-base that is cart-mounted or mounted to an operating table, and one or more manipulators mounted to the moveable base.

In various embodiments, manipulating assembly 210 has a high structural rigidity. This allows manipulating assembly 210 to reduce or eliminate motion of the RCM as described above, to achieve a high degree of kinematic precision. In some embodiments, manipulating assembly 210 is configured to limit the amounts of motive forces that can be transmitted from an actuator to a corresponding joint or link. Limits on motive forces have certain benefits (e.g., reducing a size and cost of the actuator, reducing forces exerted by manipulating assembly 210 on other devices or structures), but can also present issues. An issue is that a limit on motive force can be counterproductive in situations where larger magnitudes of joint force or torque are used to control the motion of the joint based on the commanded signals (such as when the motion of a joint or joints is to be arrested quickly, for example in response to an operator input, to avoid a collision, etc.) with quick deceleration. Desired joint force or torque commands in excess of a motive force limit saturate the actuator, which can cause jostling or overshooting of manipulating assembly 210 and/or joints and links therein, thereby causing undesirable motion of the RCM and/or reduced precision in the motion of an instrument being manipulated by manipulating assembly 210.

To address these issues, in various embodiments, manipulating assembly 210 includes one or more energy dissipative devices that are positioned to dissipate kinetic energy associated with motions along the various degrees of freedom, thereby acting as brakes to decelerate the motions and reducing saturation of the motive forces associated with the motions. As shown in FIG. 2, manipulating assembly 210 includes multiple energy dissipative devices 270, 272, 274, 276, 278, and 280. As shown, energy dissipative devices 270, 272, 274, 276, 278, and 280 are each associated with a respective one of the degrees of freedom indicated by arrows A thru F.

Energy dissipative device 270 is located on or within manipulating assembly 210 (e.g., on or within joint/link 214) to dissipate kinetic energy associated with motion along vertical adjustment A. Energy dissipative devices 272 and 274 are located on or within manipulating assembly 210 (e.g., on or within joint/link 214 or 220) to dissipate kinetic energy associated with motion along horizontal adjustments B and C, respectively. Energy dissipative devices 276, 278, and 280 are located on or within manipulating assembly 210 (e.g., on or within joint/link 220, on or within linkage 222, on or within manipulator-supporting link 224) to dissipate kinetic energy associated with yaw, pitch, and roll angular rotations D, E, and F, respectively.

In various embodiments, each of energy dissipative devices 270, 272, 274, 276, 278, and 280 can be any technically feasible energy dissipative device. Examples of energy dissipative devices include mechanical brakes, magnetic brakes, magneto-rheological fluid brakes, and/or the like. Additional examples of energy dissipative devices include adjustable dampers that can be reconfigured physically to provide variable damping.

In some embodiments, an energy dissipative device can be located physically external to a joint or physically internal within the joint. An external energy dissipative device is external to a housing of the joint and applies resistance to the motion of two or more links coupled by the joint along a degree of freedom. An internal energy dissipative device within the drivetrain applies resistance to the motion of the actuator associated with the joint along the degree of freedom. Manipulating assembly 210 can include any number and/or combination of external energy dissipative devices and internal energy dissipative device (e.g., all external, all internal, some external and some internal). In some embodiments, benefits of an external energy dissipative device include being able to engage more resistance (and thereby dissipate more kinetic energy) while limiting interference with a corresponding actuator.

In various embodiments, each of energy dissipative devices 270, 272, 274, 276, 278, and 280 are controllable by control system 170. In operation, user input system 250 receives an input from operator 298 via input controls 252, where the input indicates a target or desired motion or pose of manipulating assembly 210. Control system 170 determines, based on the current pose of manipulating assembly 210 and the target motion or pose, an amount of motive force by an actuator, whether to additionally or alternatively apply energy dissipation to the joint, and an amount of energy dissipation (e.g., braking force) by an energy dissipative device for each of one or more joints or links of manipulating assembly 210 to achieve the target motion or pose. Control system 170 can control each individual energy dissipative device to be fully or partially engaged (that is, dissipating kinetic energy at full or partial capacity, such as by fully or partially braking, or by providing maximum damping or less damping), or disengaged (that is, dissipating kinetic energy at least capacity, such as by not applying braking force. Due to physical non-idealities such as friction, a disengaged energy dissipative device may still dissipate some energy). For example, a first energy dissipative device could be controlled to be partially engaged, while a second energy dissipative device could be controlled to be fully engaged. These first and second energy dissipative devices can be applied in a temporally overlapping or non-overlapping manner. Further, in some embodiments, one or more of the energy dissipative devices are engaged and the other ones of the energy dissipative devices are disengaged during normal operation (e.g., when not in a situation of quickly arresting a motion). Further details regarding control of energy dissipative devices by control system 170 are described below.

It should be appreciated that manipulating assembly 210 can include more or fewer energy dissipative devices than as shown in FIG. 2 (e.g., manipulating assembly 210 includes energy dissipative devices for a subset of the joints or a subset of the degrees of freedom). Further, manipulating assembly 210 can include energy dissipative devices at different location(s) than those shown in FIG. 2. Also further, the repositionable structure can be of a different design or configuration from manipulating assembly 210 as shown. For example, the repositionable structure could include a side rail next to a workspace (e.g., an operating table) and manipulating arms extending from the side rail. More generally, the techniques and systems described herein can be adapted to any repositionable structure with one or more joints, including prismatic and/or rotational joints.

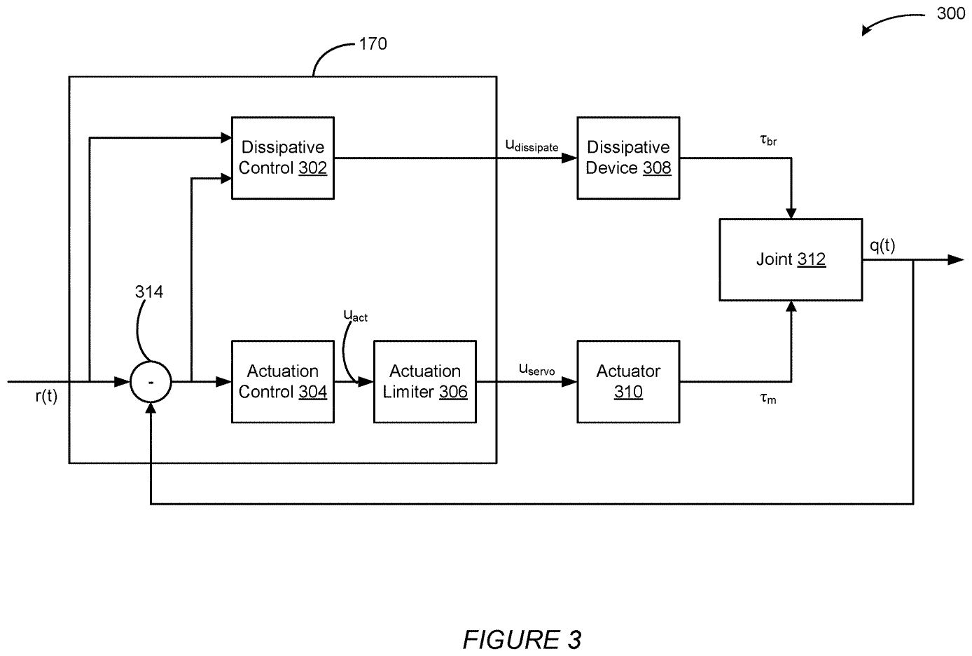

FIG. 3 illustrates a control system 300 for controlling motion of a joint and dissipating kinetic energy associated with the motion of the joint in accordance with one or more embodiments. Control system 300 includes one possible implementation for control system 170. As shown, control system 300 includes, without limitation, dissipative control 302, actuation control 304, actuation limiter 306, dissipative device 308, actuator 310, and joint 312. In some embodiments, joint 312 is a joint in a manipulating assembly 110 and/or manipulating assembly 210. In some embodiments, joint 312 is a joint in a repositionable structure of an input control (e.g., of FIG. 1) or of a user input system (e.g., part of one of repositionable structures 258 of user input system 250), such as one used to command repositionable structure 120, the manipulating assembly 210, or some other structure.”

In some embodiments, and as shown, dissipative control 302, actuation control 304, and actuation limiter 306 are implemented in control system 170. It should be appreciated that while control system 300 is illustrated with one actuator 310 and one joint 312, the components and techniques described herein with respect to control system 300 is applicable for controlling motion of, and dissipating kinetic energy associated with multiple joints in a manipulating assembly 110. For example, control system 300 can include multiple actuators 310, multiple dissipative devices 308, and multiple repositionable structure joints 312. Control system 170 can control dissipative devices 308 and actuators 310 on an individual basis (e.g., engaging dissipative devices 308 for some joints and disengaging dissipative devices 308 for other joints) in order to achieve an overall commanded or target motion for repositionable structure joints 312. Control system 170 can further be used to control dissipative devices and/or actuators in the joints of an input control, such as any of input controls 252, or a repositionable structure of an input control, such as any of repositionable structures 258.

In some embodiments, control system 170 receives one or more inputs from an operator (e.g., operator 298) via one or more input controls (e.g., input control(s) 252). The one or more inputs command a motion of manipulating assembly 110 (e.g., manipulating assembly 210). In some embodiments, the commanded motion is determined autonomously or semi-autonomously by control system 170. The commanded motion for manipulating assembly 110 is shown in FIG. 3 as r(t). In some embodiments, r(t) indicates a commanded or target position r of manipulating assembly 110 or a joint therein (e.g., joint 312) over time t. Control system 170 also receives a positional feedback of the current motion, position, or pose of joint 312, shown in FIG. 3 as q(t). In some embodiments, q(t) indicates a current position q of manipulating assembly 110 or a joint therein (e.g., joint 312) over time t.

Control system 170 determines a difference between r(t) and q(t) for joint 312 that indicates the position error 314 of the joint 312 to actuation control 304 as an input. Actuation control 304 determines an amount of actuation force or torque u_{fdbk} to be applied by actuator 310 onto joint 312 to reduce position error 314 (e.g., to cause motion of joint 312 from q(t) toward r(t)).

Actuation limiter 306 applies actuation limits onto actuator 310 by reducing the magnitude of the actuation force or torque commanded by actuation control 304 to be within an acceptable range. In some embodiments, actuation limiter 306 compares the magnitude of u_{act} to one or more predefined actuation magnitude limits, and reduces the magnitude of u_{act} to be equal to or below the magnitude limit and outputs an amount uₛₑᵣᵥₒ so that the magnitude of uₛₑᵣᵥₒ does not exceed the one or more actuation limits. For example, actuation limiter 306 can limit a magnitude of uₛₑᵣᵥₒ to be below a maximum force or torque limit. Actuation limiter 306 then generates actuation control signals for actuator 310 that commands actuator 310 to apply the force or torque uₛₑᵣᵥₒ. Control system 170 then transmits control signals corresponding to uₛₑᵣᵥₒ to actuator 310.

Dissipative control 302 determines whether to engage dissipative device 308 to dissipate kinetic energy of joint 312 (e.g., to assist actuator 310 in controlling joint 312). When dissipative control 302 determines to engage dissipative device 308, dissipative control 302 further determines an amount of dissipative force or torque u_{dissipate}, to apply using dissipative device 308 and generates control signals for dissipative device 308 that commands dissipative device 308 (e.g., energy dissipative device 270, 272, 274, 276, 278, or 280) to apply a dissipative force or torque u_{dissipate} on joint 312. Control system 170 transmits control signals corresponding to u_{dissipate} to dissipative device 308.

Dissipative control 302 first determines whether, and how, to engage dissipative device 308 to help control joint 312. Some embodiments of the dissipative device 308 provides only binary (e.g. engaged/disengaged) options, while other embodiments of the dissipative device 308 provides options for partial engagement with multiple discrete or continuous levels of engagement. Dissipative devices that provide partial engagement options may be controlled, such as by pulse width modulation over time or lowering activation voltages in spring-loaded brakes, to provide variable amounts of effective engagement. In some embodiments, dissipative control determines whether, and how, to engage dissipative device 308 based on the target motion for joint 312. In some embodiments, dissipative control 302 determines whether to engage dissipative device 308 based on one more of whether the target motion includes a deceleration of joint 312, whether the target motion includes a reversal and/or change in a current direction of motion of joint 312, a velocity of joint 312, a position of joint 312, a kinetic energy of joint 312, and/or the like. In some embodiments, dissipative control 302 determines whether to fully or partially disengage dissipative device 308 based on one or more of whether a kinetic energy of joint 312 is below a threshold, whether the target motion includes an increase in the speed of joint 312, a disabling of dissipative control (e.g., by an operator), and/or the like.

In some embodiments, dissipative control 302 and actuation control 304 work together to determine whether to control joint 312 using actuator 310 alone, using dissipative device 308 alone, or using both actuator 310 and dissipative device 308. For example, dissipative control 302 and/or actuation control 304 can determine to only use actuator 310, to only use dissipative device 308, or to use both actuator 310 and dissipative device 308. In various instances, using both actuator 310 and dissipative device 308 to control joint 312 may comprise actuating actuator 310 and engaging dissipative device 308 in a time-separated manner, such that actuator 310 is not actuated concurrently with engaging dissipative device 308, and vice versa. In various instances, using both actuator 310 and dissipative device 308 to control joint 312 may comprise actuating actuator 310 and engaging dissipative device 308 in a time-overlapped manner, such that actuator 310 is actuated concurrently with engaging dissipative device 308 for at least a period of time. For example, dissipative control 302 and/or actuation control 304 can determine to actuate actuator 310 before engaging dissipative device 308, to engage dissipative device 308 before actuating actuator 310, or to acuate actuator 310 and engage dissipative device 308 at the same time. Further example, dissipative control 302 and/or actuation control 304 can determine to stop actuating actuator 310 after stopping to engage dissipative device 308, to stop engaging dissipative device 308 after stopping to actuate actuator 310, or to stop actuating actuator 310 and stop engaging dissipative device 308 at the same time.

As shown, dissipative control 302 receives r(t) and position error 314 as inputs to determine u_{dissipate}. In some embodiments, in response to dissipative control 302 determining to engage dissipative device 308, dissipative control 302 determines u_{dissipate} according to an algorithm (e.g., an equation).

In some embodiments, u_{dissipate} is determined by Equation 1 below, where Eₖᵢₙ is the kinetic energy of joint 312, E_{kin_thresh} is kinetic energy threshold, and f is a function of one or more of the target position r of joint 312, the target velocity \dot{r}(t) of joint 312, the target acceleration \ddot{r}(t) of joint 312, the actual position q of joint 312, the actual velocity \dot{q}(t) of joint 312, the actual acceleration \ddot{q}(t) and/or other factors, such as inertial properties of joint 312 (not shown):

$${u}_{dissipate} = \{ \begin{matrix}0 & if {E}_{kin} < {E}_{kin \_ thresh} \\ f ( r , r , \ddot{r} , q , \dot{q} , \ddot{q} ) & otherwise\end{matrix} . \qquad Equation 1$$

In some embodiments, function ƒ is implemented according to Equation 2.

$$f ( r , r , \ddot{r} , q , \dot{q} , \ddot{q} ) = α \ddot{r} {I}_{axis} ( \frac{1 - sign ( \dot{r} \ddot{r} )}{2} ) . \qquad Equation 2$$

In Equation 2, 0<α<1 is a constant which modulates the contribution of Equation 2 to compensate for inertial effects during deceleration of joint 312, Iₐₓᵢₛ is an estimated rotational inertia of manipulating assembly 110 distal to joint 312 (or alternatively, an estimated mass of manipulating assembly 110 distal to joint 312, if joint 312 is a prismatic joint), and \ddot{r} is a reference command acceleration derived from r(t) (e.g., a second derivative of r(t)). Also, in some embodiments, Equation 2 as shown applies a sign function to the product of \dot{r} and \ddot{r}, which results in a non-zero u_{dissipate} (dissipative device 308 activated) during a commanded deceleration of joint 312 (e.g., when arresting motion of joint 312) and a u_{dissipate} of zero (dissipative device 308 disengaged) during a commanded acceleration of joint 312 (e.g., commanded increase in the speed of joint 312).

In an example, a could be a piecewise function based on a magnitude of a velocity tracking error (\dot{r}-\dot{q}), as shown in Equation 3 below:

$$α = \{ \begin{matrix}0 & if \vert \dot{r} - \dot{q} \vert < threshold \\ 0.6 & otherwise\end{matrix} . \qquad Equation 3$$

Inserting Equation 3 into Equations 1 and 2 results in a piecewise function for determining u_{dissipate}. Further, u_{dissipate} can be a function of one or more other parameters instead of or in addition to velocity tracking error. More generally, u_{dissipate} can be a function of r(t), a derivative of r(t), such as \ddot{r}, and/or q(t). Further, the algorithm for u_{dissipate} can be linear or non-linear.

Control system 170 then transmits control signals corresponding to u_{dissipate} to dissipative device 308.

Actuator 310 receives control signals corresponding to uₛₑᵣᵥₒ from actuation limiter 306. Based on the control signals corresponding to uₛₑᵣᵥₒ, actuator 310 actuates joint 312 with force or torque τₘ corresponding to uₛₑᵣᵥₒ to cause motion of joint 312 toward r(t). Concurrently, dissipative device 308 receives control signals corresponding to u_{dissipate} from dissipative control 302. Based on the control signals corresponding to u_{dissipate}, dissipative device 308 activates to apply a dissipative force or torque τ_{br} to joint 312 to dissipate a motive force of joint 312 by amount u_{dissipate}. Joint 312, actuated by actuator 310, and with energy dissipation applied by dissipative device 308, moves to new position q(t). New position q(t) is fed back to control system 170, where it is provided as an input into actuation control 304 and/or dissipative control 302 (e.g., indirectly via position error 314 as shown, directly into actuation control 304 and/or dissipative control 302).τ

In some embodiments, dissipative device 308 applies dissipative force or torque τ_{br} as a proportional amount of engagement of dissipative device 308. That is, dissipative device 308 activates at 100% or a proportion thereof that is appropriate to apply τ_{br} matching u_{dissipate}. Additionally or alternatively, in some embodiments, dissipative device 308 applies dissipative force or torque τ_{br} via a pulse width modulation of the dissipation control signals.

As shown, control system 170 receives q(t) as feedback input. In some embodiments, control system 170 determines and generates updated control signals to actuator 310 and dissipative device 308 based on the feedback q(t). For example, position error 314 could be updated, and actuation control 304 and/or actuation limiter 306 would generate updated actuation control signals for actuator 310 based on the updated position error 314 (e.g., control signals to actuate joint 312 with a smaller force or torque as position error 314 becomes smaller).

In some embodiments, dissipative control 302 can generate control signals to command dissipative device 308 to reduce the amount of dissipation or to even disengage dissipative device 308 in response to a magnitude of a velocity (e.g., the speed) of joint 312 decreases below a speed threshold, or more generally in response to the kinetic energy of joint 312 decreasing below an energy threshold. As used herein, the kinetic energy of a particular joint (e.g. joint 312) refers to the kinetic energy of the motion of that particular joint. For example, if a link or other physical component is being moved by the motion of that particular joint, then the kinetic energy of that entire assembly (the joint and the link or other physical component) is referred to as the kinetic energy of that particular joint. Dissipative control 302 can determine the joint speed based on q(t) throughout the motion. Based on the joint speed (or on the kinetic energy, which can be determined based on the velocity q of the joint) of joint 312, dissipative control 302 can generate updated control signals to command dissipative device 308 to dissipate a reduced amount of force or torque, or to deactivate.

Operation of manipulating assembly 110 can present various situations in which engagement of dissipative device 308 facilitates target motion of manipulating assembly 110. One example situation is in a transition between control modes (e.g., entering or exiting a clutch mode). In different control modes, the operator may be operating different sets of joints of manipulating assembly 110 (e.g., multiple joints together versus a single joint). When control of the manipulating assembly 110 transitions from a first control mode to a second control mode, motion of certain joints may need to be arrested quickly to facilitate the transition from the first control mode to the second control mode. Accordingly, dissipative device 308 can be engaged to assist in deceleration of those joints whose motions need to be arrested while reducing or eliminating undesirable motion of manipulating assembly 110.

In another example, a joint of manipulating assembly 110 can be controlling a large inertial load (e.g., a large distal mass). When the operator operates manipulating assembly 110 to reverse a motion of that joint, engagement of dissipative device 308 can facilitate quick and effective deceleration of the joint to facilitate reversal of motion, while keeping the RCM of the inertial load stationary.

In some embodiments, control system 170 can be adapted to use dissipative device 308 to provide haptic feedback to the operator on an input control, such as any of input controls 252. Control system 170 can provide haptic feedback to the operator in response to the operator driving the manipulating assembly 110 or the input control to an undesirable configuration. For example, an undesirable configuration can occur when a joint of the manipulating assembly 110 is commanded to move beyond a range-of-motion limit for the joint. Other undesirable configurations could occur when a portion of the manipulating assembly 110 is commanded to move into a keep out region, a collision or imminent collision is detected, a joint of the input control is being moved beyond a range of motion limit, and/or the like Dissipative control 302 can engage a dissipative device 308 coupled to a joint 312 of the input control to dampen and/or resist motion of joint 312 of the input control, to haptically signal to the operator that an undesirable configuration has been reached or is about to be reached. In some embodiments, u_{dissipate} for haptic feedback on joint 312 of the input control is determined using Equation 4 below:

$${u}_{dissipate} = \{ \begin{matrix}0 & q ϵ {q}_{ROM} \\ {k}_{damp} \dot{q} ( \frac{1 + sign ( \frac{d}{dt} ( \vert r ( t ) - q ( t ) \vert ) )}{2} ) & otherwise\end{matrix} . \qquad Equation 4$$

In Equation 4, k_{damp} is a dampening coefficient, which can be predefined, and \dot{q} is a velocity of joint 312 (e.g., a first derivative of q(t)). When the current position q of joint 312 of the input control is within the range of motion limit q_{ROM} for joint 312, u_{dissipate} is 0. Otherwise, u_{dissipate} is calculated based on the joint velocity, a position error between a position of joint 312 that moves joint to within the range of motion and the current position (e.g., r(t)−q(t), position error 314) and a dampening coefficient (e.g., k_{damp}). In some embodiments, dissipative device 308 is engaged concurrently for both haptic feedback and to dissipate kinetic energy. Although not shown, Equation 4 can be adapted to address other undesirable configurations by substituting tests other than qϵq_{ROM} for joint 312 that indicate whether a repositionable structure being controlled using the input control is being commanded to an undesirable configuration. In some embodiments, one or more kinematic models of the repositionable structure and the input control are used to map motion of the repositionable structure resulting in the undesirable configuration to an amount of haptic feedback to apply on the joint of the input control to resist the motion of the input control that causes motion of the repositionable structure toward or past the undesirable configuration.

Use of energy dissipative devices, as described herein, facilitates multiple benefits and advantages. One benefit is that the systems and techniques described herein better facilitate precision of motion and properly damped responses, for repositionable structures with low motive force limits and/or with joints carrying large masses or inertial loads. Another benefit is that the systems and techniques described herein facilitate higher velocity magnitudes in view of the ability to decelerate those velocity magnitudes via the energy dissipative devices. A further benefit is that the systems and techniques described herein allows for use of actuators in manipulating assembly 110 that are smaller and more compact and yet can achieve similar deceleration and arresting of motions as larger actuators, in view of energy dissipative devices aiding in deceleration and arresting of motions. Yet another benefit is that the systems and techniques described herein reduces stress and deterioration (e.g., wear and tear), and operating temperatures, of drive trains and actuators in manipulating assembly 110.

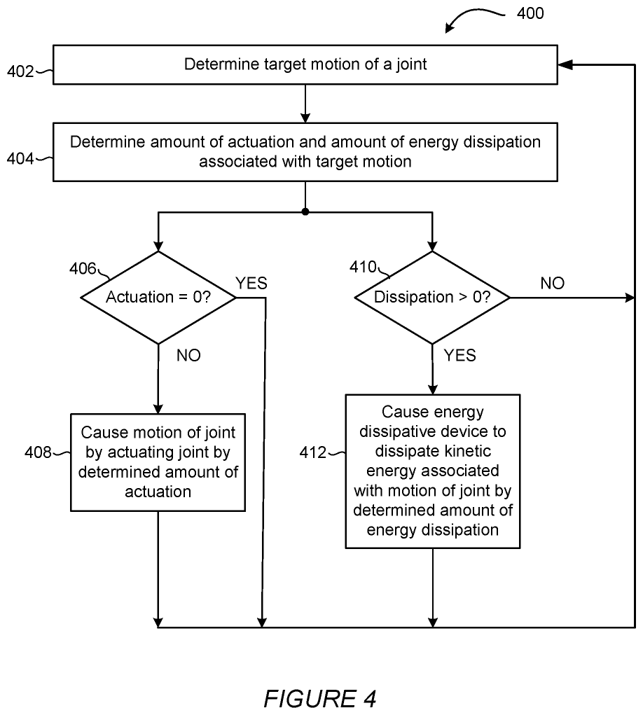

FIG. 4 is a flow diagram of method steps for dissipating kinetic energy associated with motion of a joint in accordance with one or more embodiments. Although the method steps are described with respect to the systems of FIGS. 1-3, persons skilled in the art will understand that any system configured to perform the method steps, in any order, falls within the scope of the various embodiments. In some embodiments, one or more of the steps 402-412 of method 400 may be implemented, at least in part, in the form of executable code stored on one or more non-transient, tangible, machine readable media that, when run by one or more processors (e.g., processor system 150 of control system 170), would cause the one or more processors to perform one or more of the steps 402-412. In some embodiments, steps 402-412 are performed by control system 300. As shown, method 400 shows a repeating control loop for one joint of a repositionable structure (e.g., joint 312). With each pass through steps 402-412, a target motion of the joint is updated to due to, for example, new commands for the joint, changes in the position or other kinematic properties of the joint from a previous control loop due to motion of the joint, and/or like. In some embodiments, method 400 is applied separately to each of the joints in the repositionable structure.

As shown, method 400 begins at step 402, where control system 170 determines a target motion of a joint. In some embodiments, control system 170 receives one or more inputs from an operator commanding a motion of manipulating assembly 110. In some embodiments, the target motion is determined autonomously or semi-autonomously by control system 170. Based on the inputs, control system 170 determines a target motion of a joint (e.g., r(t) of joint 312) included in manipulating assembly 110 that helps achieve the commanded motion. In some cases, the target motion includes no motion of joint 312.

At step 404, control system 170 determines an amount of actuation and an amount of energy dissipation associated with the target motion. Based on the target motion, actuation control 304 and actuation limiter 306 determine an amount of force or torque to be actuated by actuator 310. Also based on the target motion, a dissipative control 302 determines whether to engage dissipative device 308. In response to determining to engage dissipative device 308, dissipative control 302 determines an amount of dissipative force or torque to be engaged by dissipative device 308. In some embodiments, dissipative control 302 uses one or more of Equations 1-3 and/or other algorithms to determine the amount of dissipative force or torque to be engaged by dissipative device 308.

After determining the amount actuation and the amount energy dissipation in step 404, method 400 splits into two parallel paths to control the actuation of actuator 310 and/or the engagement of dissipative device 308 as described further below. In some embodiments, the path with steps 406 and 408 (actuation of actuator 310) and the path with steps 410 and 412 (engagement of dissipation device 308) can be performed in any order. For example, the path with steps 406 and 408 can be performed before, after, concurrently, and/or partially concurrently with the path with steps 410 and 412.

At step 406, control system 170 determines whether the amount of actuation determined during step 404 is equal to zero. When the amount of actuation is equal to zero (step 406—YES), no actuation of actuator 310 occurs during this pass through method 400 and method 400 returns to step 402 for the next control loop. When the amount of actuation is not equal to zero (step 406-NO), actuator 310 is actuated using step 408.

At step 408, control system 170 causes motion of the joint by actuating the joint with an actuation force or torque (e.g., a force or torque τₘ corresponding to uₛₑᵣᵥₒ). Control system 170 (e.g., using actuation control 304 and actuation limiter 306) generates actuation control signals corresponding to uₛₑᵣᵥₒ for commanding actuator 310 to actuate joint 312 by the amount of force or torque determined in step 404. Control system 170 transmits the actuation control signals to actuator 310. Actuator 310, in response to the actuation control signals, actuates joint 312 to cause motion of joint 312. After completing step 408, method 400 returns to step 402 for the next control loop.

At step 410, control system determines whether the amount of energy dissipation determined during step 404 is greater than zero. When the amount of energy dissipation is not greater than zero (step 410—NO), no engagement of dissipation device 308 occurs during this pass through method 400 and method 400 returns to step 402 for the next control loop. When the amount of energy dissipation is greater than zero (step 410—YES), dissipation device 308 is engaged using step 412.

At step 412, control system 170 causes an energy dissipative device to dissipate kinetic energy associated with the motion of the joint by the determined amount of energy dissipation. Control system 170 (e.g., using dissipation control 302) generates dissipation control signals corresponding to u_{dissipate} (e.g., u_{dissipate} determined using Equation 1 above) for dissipative device 308 to dissipate kinetic energy of joint 312 by the amount of dissipative force or torque determined in step 404. Control system 170 transmits the dissipation control signals to dissipative device 308. Dissipative device 308, in response to the dissipation control signals, activates at least partially to dissipate the kinetic energy of joint 312 as joint 312 is actuated by actuator 310. After completing step 412, method 400 returns to step 402 for the next control loop.

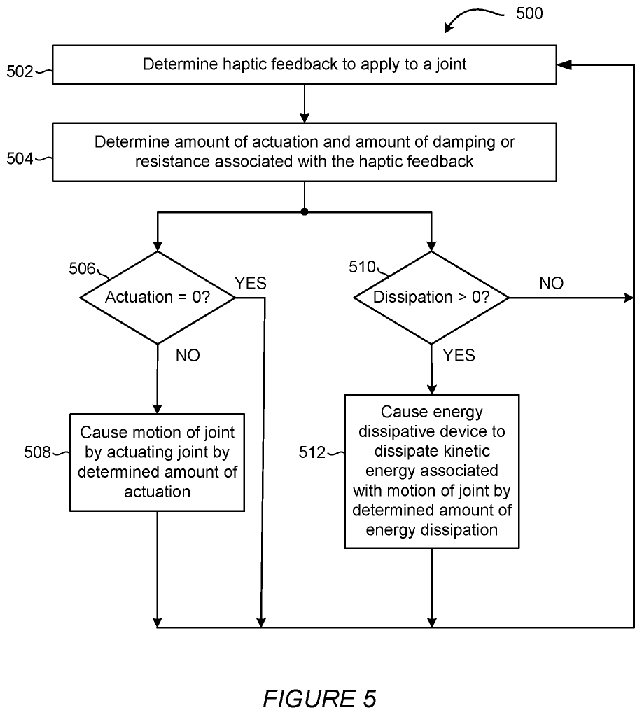

FIG. 5 is a flow diagram of method steps for applying haptic feedback to a joint in accordance with one or more embodiments. Although the method steps are described with respect to the systems of FIGS. 1-3, persons skilled in the art will understand that any system configured to perform the method steps, in any order, falls within the scope of the various embodiments. In some embodiments, one or more of the steps 502-512 of method 500 may be implemented, at least in part, in the form of executable code stored on one or more non-transient, tangible, machine readable media that, when run by one or more processors (e.g., processor system 150 of control system 170), would cause the one or more processors to perform one or more of the steps 502-512. In some embodiments, steps 502-512 are performed by control system 300. As shown, method 500 shows a repeating control loop for one joint of an input control (e.g., joint 312). With each pass through steps 502-512, haptic feedback to apply to the joint is updated to due to, for example, new levels of haptic feedback for the joint, changes in the position or other kinematic properties of the joint from a previous control loop due to motion of the joint, and/or like. In some embodiments, method 500 is applied separately to each of the joints in the input control.

As shown, method 500 begins at step 502, where control system 170 determines haptic feedback to apply to a joint. Control system 170 applies haptic feedback to the joint to provide an operator of with information that indicates that the motion of the input control being performed by the operator is to be discouraged such as might occur when the joint of the input control is at or near a range of motion limit and/or motion of the input control is causing motion in a controlled repositionable structure to an undesirable configuration. In some cases, the haptic feedback includes no haptic feedback being applied to joint 312.

At step 504, control system 170 determines an amount of actuation and an amount of damping or resistance associated with the haptic feedback. Based on the haptic feedback, actuation control 304 and actuation limiter 306 determine an amount of force or torque to be actuated by actuator 310. Also based on the haptic feedback, dissipative control 302 determines whether to engage dissipative device 308. In response to determining to engage dissipative device 308, dissipative control 302 determines an amount of dissipative force or torque to be engaged by dissipative device 308. In some embodiments, dissipative control 302 uses Equation 4 and/or other algorithms to determine the amount of dissipative force or torque to be engaged by dissipative device 308.

After determining the amount actuation and the amount damping or resistance in step 504, method 500 splits into two parallel paths to control the actuation of actuator 310 and/or the engagement of dissipative device 308 as described further below. In some embodiments, the path with steps 506 and 508 (actuation of actuator 310) and the path with steps 510 and 512 (engagement of dissipation device 308) can be performed in any order. For example, the path with steps 506 and 508 can be performed before, after, concurrently, and/or partially concurrently with the path with steps 510 and 512.

At step 506, control system 170 determines whether the amount of actuation determined during step 504 is equal to zero. When the amount of actuation is equal to zero (step 506—YES), no actuation of actuator 310 occurs during this pass through method 500 and method 500 returns to step 502 for the next control loop. When the amount of actuation is not equal to zero (step 506—NO), actuator 310 is actuated using step 508.

At step 508, control system 170 causes motion of the joint by actuating the joint with an actuation force or torque (e.g., a force or torque τₘ corresponding to uₛₑᵣᵥₒ). Control system 170 (e.g., using actuation control 304 and actuation limiter 306) generates actuation control signals corresponding to uₛₑᵣᵥₒ for commanding actuator 310 to actuate joint 312 by the amount of force or torque determined in step 504. Control system 170 transmits the actuation control signals to actuator 310. Actuator 310, in response to the actuation control signals, actuates joint 312 to cause motion of joint 312. After completing step 508, method 500 returns to step 502 for the next control loop.

At step 510, control system determines whether the amount of damping or resistance determined during step 504 is greater than zero. When the amount of damping or resistance is not greater than zero (step 510—NO), no engagement of dissipation device 308 occurs during this pass through method 500 and method 500 returns to step 502 for the next control loop. When the amount of damping or resistance is greater than zero (step 510—YES), dissipation device 308 is engaged using step 512.

At step 512, control system 170 causes an energy dissipative device to dampen or resist motion of the joint. Control system 170 (e.g., using dissipation control 302) generates dissipation control signals corresponding to u_{dissipate} (e.g., u_{dissipate} determined using Equation 4 above) for dissipative device 308 to dampen or resist motion of joint 312 by the amount of dissipative force or torque determined in step 504. Control system 170 transmits the dissipation control signals to dissipative device 308. Dissipative device 308, in response to the dissipation control signals, activates at least partially to dissipate the kinetic energy of joint 312 as joint 312 is actuated by actuator 310. After completing step 512, method 500 returns to step 502 for the next control loop.

In sum, a repositionable structure includes an energy dissipation system to assist with motion deceleration and kinetic energy dissipation. The energy dissipation system includes an energy dissipative device that can be controlled by a control unit. The control unit determines a target motion of one or more joints of the repositionable device. Based on the target motion, the control unit activates one or more actuators that control a motion of the one or more joints, and concurrently activates the energy dissipative device. The energy dissipative device assists the actuators in controlling the motion by dissipating kinetic energy associated with the motion. The energy dissipative device can be controlled to dissipate kinetic energy up to a threshold amount, after which the energy dissipative device can be disengaged.

At least one technical advantage of the disclosed techniques relative to the prior art is that, with the disclosed techniques, kinetic energy associated with motion of one or more joints of a repositionable structure can be dissipated while reducing the force and/or torque being engaged by one or more actuators associated with the one or more joints. Reducing the force and/or torque being engaged by the one or more actuators reduces wear and tear on the one or more actuators and/or allows small, less expensive, actuators to be used to control the repositionable structure. These technical advantages provide one or more technological improvements over prior art approaches.

Any and all combinations of any of the claim elements recited in any of the claims and/or any elements described in this application, in any fashion, fall within the contemplated scope of the present disclosure and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Further, the terminology in this description is not intended to limit the invention. For example, spatially relative terms-such as “beneath”, “below”, “lower”, “above”, “upper”, “proximal”, “distal”, and the like—may be used to describe the relation of one element or feature to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, or module may, whenever practical, be included in other embodiments, implementations, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

While the preceding is directed to embodiments of the present disclosure, other and further embodiments of the disclosure may be devised without departing from the basic scope thereof, and the scope thereof is determined by the claims that follow.

1. A computer-assisted system comprising:
a repositionable structure comprising: a first joint, a first actuator configured to control motion of the first joint, and a first energy dissipative device, wherein engaging the first energy dissipative device dissipates a kinetic energy of the first joint; and
a control system coupled to the repositionable structure;
wherein the control system is configured to:
determine a first target motion for the first joint,
determine, based on at least the first target motion, whether to concurrently actuate the first actuator and engage the first energy dissipative device such that the first energy dissipative device dissipates the kinetic energy of the first joint while the first actuator drives motion of the first joint, and
in response to the determination to concurrently actuate the first actuator and engage the first energy dissipative device, concurrently actuate the first actuator and engage the first energy dissipative device.

2. The computer-assisted system of claim 1, wherein to determine whether to concurrently actuate the first actuator and engage the first energy dissipative device, the control system is configured to:
determine whether the first target motion comprises decelerating the first joint.

3. (canceled)

4. The computer-assisted system of claim 1, wherein to determine whether to concurrently actuate the first actuator and engage the first energy dissipative device, the control system is configured to:
determine whether the first target motion comprises a change in a current direction of motion of the first joint.

5. (canceled)

6. The computer-assisted system of claim 1, wherein the control system is further configured to:
in response to a determination that, after concurrently actuating the first actuator and engaging the first energy dissipative device, a speed of the first joint is below a speed threshold or the kinetic energy of the first joint is below an energy threshold, at least partially disengage the first energy dissipative device.

7. (canceled)

8. The computer-assisted system of claim 1, wherein:
the repositionable structure further comprises: a second joint, a second actuator configured to control motion of the second joint, and a second energy dissipative device, wherein engaging the second energy dissipative device dissipates a kinetic energy of the second joint; and
the control system is further configured to:
determine a second target motion for the second joint,
determine, based on the second target motion, whether to concurrently actuate the second actuator and engage the second energy dissipative device such that the second energy dissipative device dissipates the kinetic energy of the second joint while the second actuator drives motion of the second joint, and
in response to the determination to concurrently actuate the second actuator and engage the second energy dissipative device, concurrently actuate the second actuator and engage the second energy dissipative device.

9. The computer-assisted system of claim 1, wherein the first energy dissipative device comprises a mechanical brake, a magnetic brake, or a magneto-rheological fluid brake.

10. The computer-assisted system of claim 1, wherein the first energy dissipative device comprises a damper, and wherein the control system is configured to engage the first energy dissipative device by increasing a damping of the damper.

11. The computer-assisted system of claim 1, wherein to concurrently actuate the first actuator and engage the first energy dissipative device, the control system is configured to:
determine, based on the first target motion and a velocity or position of the first joint, an amount of energy to dissipate by engaging the first energy dissipative device.

12. The computer-assisted system of claim 1, further comprising an input device configured to receive input from an operator, wherein the control system is further configured to:
receive a motion command for the repositionable structure from the input device; and
determine the first target motion based on the motion command.

13. (canceled)

14. The computer-assisted system of claim 1, wherein the control system is further configured to:
after concurrently actuating the first actuator and engaging the first energy dissipative device and in response to a command to increase a speed of the first joint, disengage the first energy dissipative device.

15. The computer-assisted system of claim 1, wherein the control system is further configured to:
determine, based on an acceleration associated with the first target motion or the kinetic energy of the first joint, an amount of dissipative force or torque to apply to the first joint using the first energy dissipative device; or
determine, based on the kinetic energy of the first joint, the amount of dissipative force or torque to apply to the first joint using the first energy dissipative device.

16. (canceled)

17. The computer-assisted system of claim 1, further comprising:
an input control comprising a third joint, a third actuator configured to control motion of the third joint, and a third energy dissipative device;
wherein:
engaging the third dissipative device dampens motion of the third joint;
the input control is configured to be usable by an operator to control the repositionable structure; and
the control system is further configured to:
determine that haptic feedback is to be applied to the third joint,
determine, based on at least the haptic feedback, whether to concurrently actuate the third actuator and engage the third energy dissipative device such that the third energy dissipative device resists motion of the third joint while the third actuator drives motion of the third joint, and
in response to the determination to concurrently actuate the third actuator and engage the third energy dissipative device, concurrently actuate the third actuator and engage the third energy dissipative device.

18. (canceled)

19. A method comprising:
determining, by a control system, a first target motion for a first joint of a repositionable structure;
determining, by the control system based on at least the first target motion, whether to concurrently actuate a first actuator and engage a first energy dissipative device such that the first energy dissipative device dissipates kinetic energy of the first joint while the first actuator drives motion of the first joint; and
in response to the determining to concurrently actuate the first actuator and engage the first energy dissipative device, concurrently actuating, by the control system, the first actuator and engaging, by the control system, the first energy dissipative device.

20. The method of claim 19, wherein determining whether to concurrently actuate the first actuator and engage the first energy dissipative device comprises:
determining whether the first target motion comprises decelerating the first joint.

21. (canceled)

22. The method of claim 19, wherein determining whether to concurrently actuate the first actuator and engage the first energy dissipative device comprises:
determining whether the first target motion comprises a change in a current direction of motion of the first joint.

23. (canceled)

24. The method of claim 19, further comprising:
in response to determining that, after concurrently actuating the first actuator and engaging the first energy dissipative device, a speed of the first joint is below a speed threshold or the kinetic energy of the first joint is below an energy threshold, at least partially disengaging, by the control system, the first energy dissipative device.

25-29. (canceled)

30. The method of claim 19, further comprising:
receiving, by the control system, a motion command for the repositionable structure from an input device configured to receive input from an operator; and
determining, by the control system, the first target motion based on the motion command.

31-32. (canceled)

33. The method of claim 19, further comprising:
determining, by the control system based on an acceleration associated with the first target motion or the kinetic energy of the first joint, an amount of dissipative force or torque to apply to the first joint using the first energy dissipative device.

34. (canceled)

35. The method of claim 19, further comprising:
determining, by the control system, that haptic feedback is to be applied to a third joint of an input control configured to be useable by an operator to control the repositionable structure;
determining, by the control system based on at least the haptic feedback, whether to concurrently actuate a third actuator and engage a third energy dissipative device such that the third energy dissipative device resists motion of the third joint while the third actuator drives motion of the third joint; and
in response to determining to concurrently actuate the third actuator and engage the third energy dissipative device, concurrently actuating, by the control system, the third actuator and engaging, by the control system, the third energy dissipative device.

36. (canceled)

37. One or more non-transitory machine-readable media comprising a plurality of machine-readable instructions which when executed by a processor system associated with a computer-assisted system are adapted to cause the processor system to perform a method comprising:
determining a first target motion for a first joint of a repositionable structure;
determining, based on at least the first target motion, whether to concurrently actuate a first actuator and engage a first energy dissipative device such that the first energy dissipative device dissipates kinetic energy of the first joint while the first actuator drives motion of the first joint; and
in response to the determining to concurrently actuate the first actuator and engage the first energy dissipative device, concurrently actuating the first actuator and engaging the first energy dissipative device.
